(19) United States
(12) Patent Application Publication
(10) Pub. No.: US 2025/0228652 A1
(43) Pub. Date: Jul. 17, 2025

(54) Dental Bar Implant and Overdenture and Process for Using.

(71) Applicants: Mayank R. Adatia, Naperville, IL (US); Anand Adatia, Naperville, IL (US); Shakuntala M. Adatia, Naperville, IL (US); [Neha] [Morker], Oak Brook, IL (US)

(72) Inventors: Mayank R. Adatia, Naperville, IL (US); Anand Adatia, Naperville, IL (US); Shakuntala M. Adatia, Naperville, IL (US); [Neha] [Morker], Oak Brook, IL (US)

(21) Appl. No.: 18/751,328

(22) Filed: Jun. 23, 2024

Related U.S. Application Data
(60) Provisional application No. 63/620,793, filed on Jan. 13, 2024.

(51) Int. Cl. A61C 13/10; A61C 13/271

(52) U.S. Cl. CPC A61C 13/1003; A61C 13/26

(57) ABSTRACT

A two-part denture and inlay for implanting within the jaw of an edentulous patient thereby restoring the aesthetics and ability of the patient to eat. The denture has a blade-like lower edge that fits into a corresponding groove in the upper surface of the inlay, in a friction-fit manner, so that the patient cannot easily remove the denture. However, using screws in threaded holes provided in the denture, the dental practitioner can disengage the denture from the inlay and replace the denture, when necessary. Also, a method of using the two-part denture and inlay is provided.

### BACKGROUND OF THE INVENTION

The subject invention addresses four major goals in the field of dentistry, in particular, in the field of dental implantology (i.e., dental implant-and-denture appliances and their installation in a patient's jaw by a licensed dental practitioner). The four major goals include: a) low relative cost of the preparation of and installation of the implant-denture appliance in the edentulous or partially edentulous patient (i.e., resulting in lower overall cost), b) rapid speed of manufacture and rapid speed of installation of the appliance (e.g., within or near the dental practitioner's place of practice, and within days or within a single day of the patient's visit), c) personalized (or “precision”) dental care (e.g., replaceable dentures (i.e., “overdentures”), that accurately match the appliance with the patient's jaw features, meeting aesthetic requirements, etc.), and, d) providing the ability to easily replace the denture as necessary, e.g., due to routine wear, etc.

The core feature of the subject invention is the unique relationship between the overdenture (“denture”) and the implant (i.e., the resulting combination of the overdenture-and-implant “appliance”), in which the appliance has an overall generally arcuate bar shape that corresponds to the corresponding arcuate shape of the edentulous portion (at least two or more contiguous missing teeth, rather than a single tooth) of the patient's jaw, and, in particular, the overdenture has a generally arcuate bar-shaped base portion (i.e., the overdenture “blade”) that matches a corresponding generally arcuate groove in the surface portion of the implant in a conforming interference fit of the blade within the groove.

When fully installed, the base portion (blade) of the overdenture mates securely within the groove portion of the implant to form a tight interference fit, so that, after installation of the appliance, the overdenture can only be separated from the implant by the dental practitioner using a set of screws that, by threading the screws through threaded holes in the overdenture, thereby overcomes the pull-force needed to separate the overdenture from the implant and causing the overdenture to thereby separate from the implant. A replacement overdenture can then be installed in the existing implant that remains in the jaw of the patient.

The field of dentistry is familiar with interference fit relationships between conically shaped implants for individual teeth (usually threaded into the jaw bone). Note: the term “interference fit” is equivalent to the terms: “pressure fit”, “press fit”, “friction fit” and other similar terms for using the friction engineering concept of mating two objects by applying the well-known “Coulomb Law of Friction” (in the field of statics and mechanics) that provides the relationship between the amount of force needed to initiate movement between two stationary surfaces that are initially in full static contact; this relationship establishes the concept of “friction coefficient”.

In the subject invention, however, the “interference fit” occurs between the lower blade portion of the overdenture and the corresponding upper groove portion of the implant, rather than between a conically shaped abutment in a similarly conically shaped implant. The field of dentistry is also familiar with using two or more of the individual conically shaped implants to support an arcuate bar-shaped denture that is attached to the jawbone using the conical implants, generally for permanent installation in the patient's jaw. However, these individual conical implants are not designed for easy removal of and replacement of the overdenture as provided for in the subject invention.

In order to produce the subject implant and overdenture, well-known and accurate 3D measurements are made of the patient's jaw (particularly the edentulous portion of two or more contiguous missing teeth of the patient's mandible or maxilla), to determine all parameters needed for the dental practitioner to prepare the personalized overdenture-and-implant appliance for the patient.

These 3D measurements can be made with any 3D scanner that provides sufficient accuracy (e.g., near or below 1 micron) of all tissue and existing implants in the patient's jaw. A wide variety of technologies exist for making the initial 3D dental image of the patient's jaw, including the edentulous areas. For example, oral scanners, CBCT (cone beam computed tomography) scanners, panoramic scanners, high-resolution cameras and laser scanners all exist that can generate an accurate 3D digital image (e.g., in STL format) of the patient's jaw. The STL image can be converted into a DICOM digital image for use in the dental milling/manufacturing equipment.

The 3d scanner must be able to produce an accurate 3D digital image that can be transmitted to a 3D printer (to make precision surgical guides for the dental practitioner to use to make accurate cuts and preparation of the jaw for the implant, and to make part of the overdenture), and to a precision milling system (to manufacture the implant and base portion of the overdenture). A common 3D image format is STL. However, other digital formats are also available and may have different resolutions and compatibilities with the 3D printer and/or precision dental milling machine used, including OBJ and PLY. Another format is DICOM that can be used with STL to provide digital images that provide information for the 3D printing and dental milling machines. The article “Accuracy of DICOM-DICOM vs DICOM-STL Protocols in Computer-Guided Surgery: A Human Clinical Study”, Journal of Clinical Medicine, MDPI, published Apr. 22, 2022, describes some important details regarding the accuracy of using various digital technology in making dental implants for edentulous patients. The milling equipment can also include capabilities for various additional treatments including polishing, etching, surface coating, etc. that are all well known in the field of dental implantology, especially, in the case of the subject invention, when applied to ensure that the resulting mating surfaces of the overdenture blade and corresponding implant groove have the necessary strength of the resulting interference fit.

The following is a list of youtube videos that show various well-known and recent procedures and equipment used for making conventional dental implants, including dental bars that are installed using screws and abutments, and using Morse taper shapes that allow an interference fit between two conically shaped parts. However, none of these show the particular blade and groove interference-fit relationship of the subject invention:
“Why Neodent GM Helix for Dental Implants-UezD1w4_UP4.mp4” (shows use of Morse taper in conically shaped implants and abutments)
“Titanium Custom Abutment Milling-ApEk-K2QDrQ.mp4” (shows a dental milling machine for use in computer-controlled making of a titanium abutment)
“Dental Milling CAD_CAM—Continuous 5 Axis-vS2Tm4U3RJQ.mp4” (shows another model dental milling machine that uses digital 3D image for computer controlled milling)
“3Shape Dental System—Implant Bar Design-NjeTpoQd3bc.mp4” (shows another dental system for automatically machining dental implants—note: the bar is attached to the patient's bone using screws)

The 3D image is then modified to incorporate a digital arcuately shaped bar implant within the digital edentulous portion (two or more contiguous teeth) of the patient's jaw. In the subject invention, in particular, an overdenture having a blade-like base portion that matches a corresponding groove in a surface portion of the implant, is also digitally added to the 3D image. One or more surgical guides are also digitally added to the image that conform to the particular 3D geometry of the edentulous areas of the patient (i.e., including surrounding soft tissue/gingiva, etc.). The surgical guides are used by the dental practitioner to ensure that the surgical tools used are limited in their movement through the alveolar ridge cortical bone, and the depth of the tool in the medullary bone, thereby, for example, preventing overcutting, or puncturing through the nasal sinus lining.

The process of preparing the edentulous area of the maxillar or mandibular alveolar ridge for the subject overdenture and implant appliance, includes cutting a strip of the alveolar ridge to expose the medulary bone beneath, a common technique in conventional dental implant practice. A striplike portion of the medullary bone is then compacted with an appropriate surgical tool (e.g., a high frequency compactor) to initiate the natural process of osseodensification. In the subject invention, the removed strip of cortical bone and a portion of the medullary bone is then crushed and mixed with a growth medium that will be placed back into the channel-like exposed medullary bone. This bone-growth medium mixture will ensure the rapid regeneration of needed tissues, such as blood vessels, nerves and other tissues that provide necessary structure and biocompatibility of the patient's medullary bone tissue with the implant, as well as accelerating the processes of initial osseodensification of the medullary bone and subsequent osseointegration of the implant with the surrounding medullary bone.

Then the implant is installed into the exposed medullary bone and tapped in to ensure firm mechanical contact with the surrounding edges of the opening that was formed in the cortical portion of the alveolar ridge, thereby initiating the natural process of osseointegration of the implant with the surrounding enhanced medullary bone tissue.

After the implant has been firmly seated in the medullary bone of the edentulous area of the patient's jaw, the overdenture is then installed onto the implant, i.e., the blade portion of the overdenture mates firmly with the corresponding groove of the implant and held firmly in place by the resulting interference fit that takes place. A suitable tool, e.g., a dental mallet or similar tool that can apply sufficient force (e.g., tapping forces) to ensure that the overdenture mates fully with the implant, e.g., strong enough to prevent reaching a minimum “pull-force” needed to separate the two parts of the appliance. This “pull-force” must be sufficiently high to prevent the patient from being able to separate the overdenture from the implant during normal use. If (or when) the overdenture needs to be replaced, which can occur about every 1-10 years of routine use, the dental practitioner can use custom screws that fit into threaded holes in the overdenture, that, when threaded into the holes, will force the overdenture to separate from the impant. After the overdenture is removed, the implant is then prepared (e.g., cleaned, polished) before installing a replacement overdenture. A replacement overdenture is then manufactured and installed in the existing implant.

In the event that the patient's edentulous areas are insufficient to properly receive the implant and overdenture, the dental practitioner can use well known augmentation procedures to bring the edentulous area up to the necessary requirements for the installation of the appliance. Such procedures can include sinus lift, bone grafting, etc., which are well known in the field of dental implantology.

The materials used for the subject appliance include both well-known existing metals, alloys, ceramics, as well as state-of-the-art dental materials that meet the following requirements: a) biocompatibility with the surrounding medullary bone tissue and resistance to attack by various biological agents (including allergic reactions); b) sufficient angle of the outer sides of the blade-like lower portion of the overdenture and corresponding identical angle in the inner sides of the groove portion of the implant to establish a sufficient “mating grip” that holds the appliance together (i.e., provides sufficient friction force to prevent the patient from separating the overdenture from the implant during normal use, including an appropriate safety factor), and c) sufficient mechanical strength to withstand routine masticatory forces that can take place.

Existing, known materials for the implant and for the “lower” portion of the overdenture that mates with the implant include: Titanium, Zirconia, Cobalt-Chromium, as well as Titanium alloys. The 3D printed surgical guides can be made using PMMA (polymethylmethacrylate) and similar 3D printing materials that provide sufficient strength and accuracy in manufacture.

All of the steps required to perform the entire process, from initial consultation and 3D imaging of the patient's jaw, the modification of the initial 3D digital image to include one or more surgical guides and the two-part appliance, to the actual manufacture of the custom-built surgical guides and the custom-built dental appliance parts and their installation in the patient's jaw, can be performed within a single facility that is staffed and equipped appropriately, including the certified dental practitioner who coordinates the entire process.

Therefore, the cost can be reduced by using nearby manufacturing equipment and having a suitable supply of milling blanks and material for making the surgical guides and appliance; the time can be reduced by eliminating the turn-around needed to send out to remote manufacturing facilities to make parts; the resulting appliance is custom-built for correcting the particular edentulous area (i.e., two or more contiguous missing teeth) of the patient; and the overdenture can be easily replaced as needed by the dental practitioner.

### SUMMARY OF THE INVENTION

The invention is a 2-part inlay-denture combination structure and a method of using the combination. More specifically, the inlay part (Component-X) is usable as a dental implant installed within an arbitrary arcuate length of a contiguous edentulous region of the jaw of a patient. The arcuate shape of the inlay corresponds generally to the shape of a portion of or an entire arch of edentulism (in this case, any two or more neighboring/contiguous missing teeth) in a patient. The second part is a denture base, Component-Y, with attachment features provided on its coronal surface to which prosthetic teeth are permanently attached. The apical edge of the entire length of the denture base has a blade shape in which each of the sides of the blade have a surface that slants from a wider blade thickness to a narrower thickness (along the coronal to the apical direction) along the entire width of the apical blade edge (between the distal ends), thereby forming a smooth 2-9° slant (corresponding to a “Morse taper”) with respect to the vertical on both sides of the blade. The inlay part, Component-X, has a groove extending along its entire arcuate length that also has inner side surfaces along the groove that are slanted from a wider groove width at the coronal surface of the inlay towards a narrower width along the apical edge of the inlay, in a smooth manner. When the denture is fully installed into (and onto) the inlay, the apical blade portion of the denture base fits into the groove of the inlay and forms a mated 2-part structure held together by friction fit. This friction fit causes the denture to be held onto the inlay in a sufficiently strong manner to prevent the removal of the denture by the patient during normal use.

The inventive method of using the 2-part inlay-denture structure involves the surgical preparation of the edentulous portion of the jaw to receive the inlay, the installation of the inlay, and the installation of the denture structure (including attached prosthetic teeth) onto the inlay (creating a 2-part assembly that is held together by friction forces), in which the final installed 2-part dental appliance is held securely within the jaw of the patient and can immediately be loaded (i.e., the patient can immediately use the installed appliance for eating).

### DESCRIPTION OF THE DRAWINGS

FIG. 1 shows a perspective view of the inlay (bottom) and denture base (top).

FIG. 2 shows an elevation cross-sectional view of the inlay and denture joined together (by Morse taper friction fit) and fully installed within the alveolar process (in this case, the mandible).

FIGS. 3A and 3B show a diagram of the general work flow for making the X- and Y-components of the invention from titanium blanks using a combined STL file and a CNC automated milling machine, and a surgical guide using the combined STL file and a 3D printer.

FIG. 4 shows an elevational cross-sectional view of the denture base at the position of a screw-hole in which a screw is used for disengaging the X-component from the Y-component, e.g., for replacing the denture.

### DETAILED DESCRIPTION OF THE INVENTION

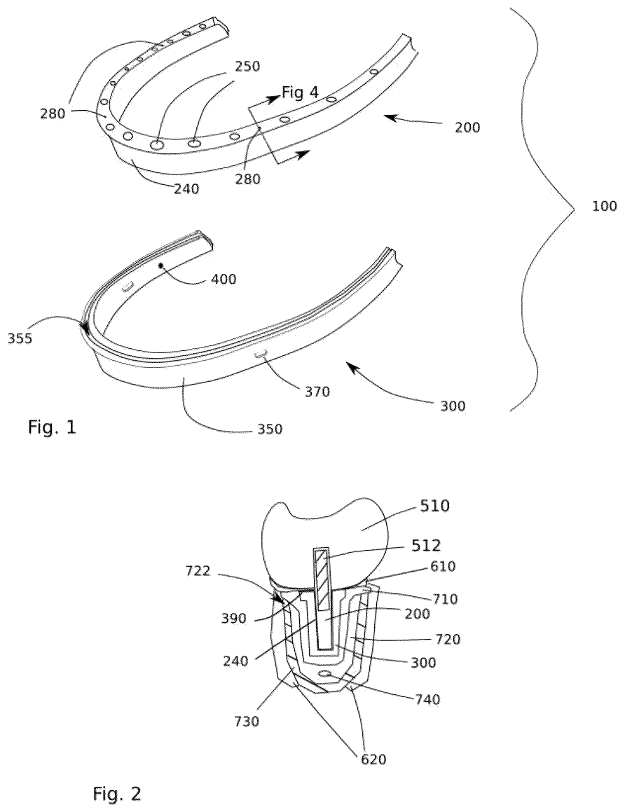

FIG. 1 shows a perspective view of the inventive 2-part inlay-denture structure (i.e., an X-component and a Y-component, respectively) 100 which are installed within a corresponding alveolar process of either the mandible or maxilla, after the alveolar process (730, FIG. 2) has been surgically prepared. Although the figure shows a full arch structure for these two parts, a partial arch is also contemplated, depending on the extent of edentulism that the patient requires to be corrected by the installation of the inventive 2-part structure.

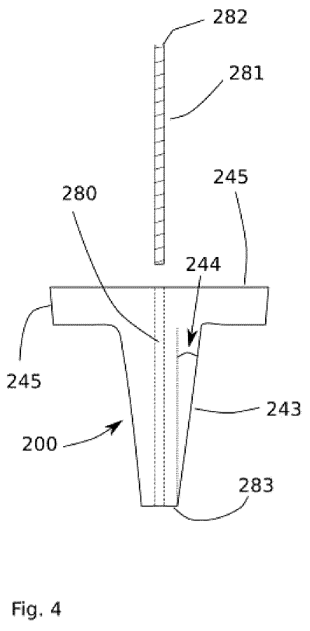

The denture base (the Y-component) 200 has a plurality of attachment locations 250 provided (the number of attachment locations corresponding to the number of prosthetic teeth, not shown, in FIG. 1, being provided to the edentulous, or partially edentulous, patient). The apical edge 240 of the denture base 200 is bladelike and has converging tapered sides 243 as shown in the cross-sectional view of FIG. 4. The angle 244 of the tapered sides are relative to the vertical direction. This angle is called a Morse taper and can vary from 2-9°. A groove 355 (FIG. 1) is provided along the entire length of the arcuately shaped inlay 300. The inner sides of this groove 355 are also tapered with the same angle as used on the sides of blade portion of the denture base 240, to approximate accurately to the counterpart.

The inlay 300, FIG. 1, is provided with one or more horizontally positioned ribs 370 along the outer surface 350 of the inlay, which help prevent dislodgement or easy movement of the inlay along the vertical (axial) direction, after installation.

After surgical preparation of the jaw is complete, the inlay is installed into the jaw, the denture (including the base and all locations 250 with attached prosthetic teeth) is then press fit into the corresponding groove of the inlay. This provides excellent resistance to all deflecting forces during the mastication cycles. The resulting friction fit prevents the removal of the denture from the inlay during use, until the denture is intentionally removed by the dental practitioner, e.g., for replacement with another denture.

FIG. 2 shows a cross-sectional view of the inventive 2-part inlay-denture structure, after being fully installed. When fully installed in the patient's jaw, the denture base 200 has coronal edges (245, FIG. 4) that are in close proximity with, or in direct contact with, the corresponding coronal edge of the alveolar crest (622, FIG. 2). Prosthetic teeth 510 are attached to the denture base 200 using posts 512 or any other suitable means that fixes the prosthetic teeth to the denture base until the denture is removed and replaced at some later time by the dental professional with a replacement denture. The dentist can disengage the counterparts X and Y by simple rotation of the disengaging screws 281 (FIG. 4). The coronal surface 390 of the denture base 200 has a reduced width relative to the apical surface of the prosthetic teeth 510 and the attachment post 512 and represents “platform switching” (i.e., using a smaller ‘platform surface’ than the size of the attachment/abutment) that has been found to enhance the maintenance of healthy mucogingival junction recovery of the gingival and mucosal tissue 610, 620 after installation of the inlay-denture structure.

The overall construction of the inlay-denture structure 100, FIG. 1, begins with accurate dental imaging by the dental professional, using an image of the edentulous area of the patient, produced by a CBCT (cone beam computed tomography) camera, a well-known contemporary tool, and an intraoral scan image made using an intraoral scanner camera (also well-known tool). The digital image of the CBCT scanner and the digital image from the intraoral scanner are then digitally combined to produce an accurate 3D STL file (“stereolithgraphy” file format used in 3D printing, rapid prototyping, and computer-aided manufacturing, such as CNC, computer numerical control manufacturing).

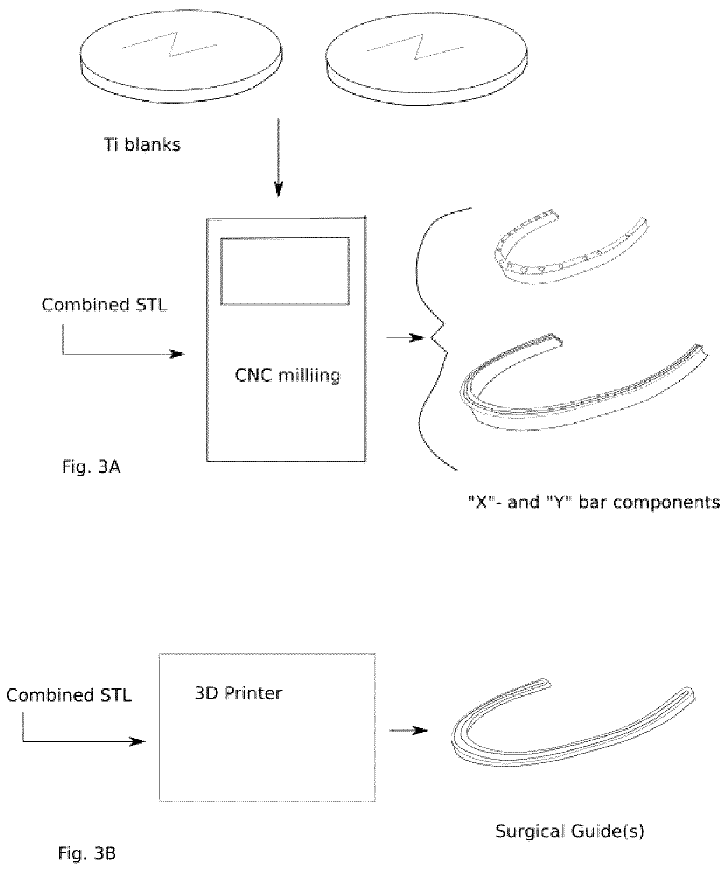

After the final 3D STL file has been digitally created (by combining the CBCT and intraoral scanner images), this STL file is then used in a CNC machine to form a titanium inlay and denture base from titanium blanks as shown in FIG. 3A. The same combined STL file is used in a 3D printer to make surgical guides that the dental professional then uses to prepare the jaw of the patient prior to installing the inlay and denture, as shown in FIG. 3B. These techniques and machines are well-known to dental professionals. The surgical guide (or guides) simplifies the site preparation by precisely controlling the depth and direction of the surgical tools used to form the channel within the alveolar bone as determined by the digital planning in advance. The guides are used with corresponding surgical tools to guide the path of the incision made by the dental professional in the edentulism region of the jaw of the patient. The guide(s) limit the width and depth of the incision by providing suitable ‘stops’ that limit the movement of the tools, thereby making sure the intended incision is accurately made for the specific inlay and denture components (which are unique to the patient) to be properly installed.

The dental practitioner will place the surgical guide onto the patient's gums along the arcuate path of the edentulous area of the jaw. A first incision is made to remove the overlying gingiva and mucosal soft tissue. Then, using the same (or another) surgical guide, the dental practitioner cuts a path in the cortical bone along the alveolar crest corresponding to the region where the inlay and denture will be installed. The surgical guide limits side-to-side and depth movements of the cutting tools used. Once the cortical bone has been removed, the next step is to create a channel along the arcuate path through the alveolar crest (722, FIG. 2), using a drill equipped with burs. The drill and burs are moved along the entire arcuate path in the alveolar cortico-cancelous bone tissue to form a channel for receiving the inlay. The dental practitioner performs this movement along the same path a second time, but this time with the drill rotating in the opposite direction, thereby initiating osseodensification of the cortico-cancelous tissue along the channel. The collected autogenous bone (cortico-cancellous tissue) is mixed with A and I PRF (advanced and injectable platelet-rich fibrins) and preferred augmentation grafts, and the resulting mixtures are condensed with special piezo compactor tip surgical tools to achieve the necessary osseodensification within the channel (710, FIG. 2) formed in the cancelous bone tissue (720, FIG. 2) in the alveolar bone 730, to complete the recipient site for the Component-X installation. The surgical guide(s) prevent damage during preparation of the recipient site channel, to critical anatomical structures, such as the nerve-vessel bundle in the apical region of the jaw (740, FIG. 2)

The inlay, Component-X, is then installed into the channel formed in the alveolar process. Finally, the denture is installed onto the inlay, with the apical blade of the denture base fitting exactly within the corresponding groove of the inlay, and a surgical tool (e.g., piezoelectric or pneumatic hammer/mallet) is used to force the denture into a friction fit with inlay so that the patient cannot remove the denture. The overdenture with the Component Y is thus press-fitted into the Component X, and the articulation is adjusted in vivo as needed. After final installation of the denture onto the inlay, the coronal outer edges (245, FIG. 4) are even with and in close proximity with or in direct contact with the coronal edges of the alveolar crest (FIG. 2).

The entire process, from making the digital images with the CBCT and intraoral cameras to the completion of installation of the inlay and denture, is anticipated to take one visit to the dental practitioner. Costs are significantly reduced and accuracy is increased with the automated processing steps. After installation, the patient can ‘load’ the dentures, i.e., eat foods without requiring temporary prosthodontal appliances, and can immediately adapt to gradually vigorous chewing.

The overdentures (dentures) can be serviced or replaced as required and replacement prostheses can be fabricated effortlessly in advance from the digital files by the lab, as necessary.

When the denture wears, after some extended period of use, e.g., 1-5 years, the denture can be replaced. This is performed by the dental practitioner during a subsequent visit, using a set of screws (one shown 281, FIG. 4) that are screwed into corresponding threaded holes 280 provided in the denture 200, FIG. 1, that span the height of the denture, from its coronal surface 245 to the apical surface 283, as shown in FIG. 4. The top of the screw 282 can be any known type, e.g., flat-head, Philips, Torx, etc. When the dental practitioner has turned the screws sufficiently to extend the apical tips of the threaded holes (283) into contact with the coronal surface of the underlying inlay, the denture will separate from the inlay, and a new denture can then be installed (made from a new set of CBCT/intraoral scans and a resulting new STL file, to make a new dental base with new prosthetic teeth attached).

Biocompatibility of titanium has been well established for surgical implants and several bacteriostatic coatings are routinely used in surgical procedures.

In addition, the inlay 300 can be provided with a communication port 400, FIG. 1, and an internal glucose sensor and digital circuitry (eg., data storage, wireless communication with an external reader) to make on-demand and continuous glucose level measurements of the interstitial fluid in the osseodensified and compacted cancelous tissue surrounding the inlay. The glucose sensor can involve an electrochemical measurement or can use a radiative signal, e.g., near infrared (NIR) measurement, to determine the interstitial glucose level, as is known in the field of implants for physiological measurements. The use of an on-demand or continuous glucose measurement within the oral cavity can be useful to patients who have diabetes (or pre-diabetes) and must control their glucose levels. The patient can then change their eating habit to restore their glucose levels to a “good range” of values.

Instant blood sugar spikes alerts with the endosseous port can help control hypoglycemic fatalities and enhance patient awareness of the less desired food intake to control pre-diabetic conditions.

1. A two-part dental appliance for use in installing within an arcuate portion of a maxilla or mandible of a jaw of a partially or completely edentulous patient, the appliance comprising:
an arcuately shaped inlay having an outer width, an outer height, a top surface, a bottom surface, an arc-length, and two distal ends, and,
an arcuately-shaped denture base having an outer width, a top surface, a bottom surface, an arc-length, and two distal end-walls, the arc-length of of the arcuately-shaped denture base corresponding to the arc-length of the inlay;
wherein the inlay has a groove extending along the entire arc-length of the inlay, the groove having an internal circumferential wall extending along the entire circumference of the groove, the groove further having a groove depth and a groove bottom, wherein the internal circumferential wall comprises an inner arcuate wall, an outer arcuate wall opposing the inner arcuate wall, a groove thickness between the two arcuate walls, two opposing distal walls, and a groove arc-width between the two distal walls, thereby enclosing the circumference of the groove,
wherein all of the inner arcuate wall, outer arcuate wall, and both distal walls of the groove have a smooth slant with respect to the vertical direction, convergence of 2-9 Degrees extending from the top surface of the inlay to the groove bottom, whereby the width and thickness of the groove at the top surface are larger than the width and thickness of the groove at the groove bottom, respectively,
wherein the arcuately-shaped denture base has multiple prosthodontic teeth attached to the top surface that correspond to the positions of missing teeth in the edentulism of the maxilla or mandible of the patient,
the arcuately-shaped denture base further having a blade-shaped bottom edge extending along the bottom of the entire arc-length of the arcuately-shaped denture base, the blade-shaped bottom edge having two opposing arcuate sides, a height, a thickness between the two arcuate sides, two opposing distal end surfaces, and an arc-width between the distal end surfaces,
the opposing sides of the blade-shaped bottom edge and the distal end-walls, each having a smooth slant with respect to the vertical direction, such that the width and thickness of the blade-shaped bottom edge decreases vertically from the top surface toward the bottom surface;
wherein the height, thickness and arc-width of the blade-shaped bottom edge of the arcuately-shaped denture base corresponds in size to the groove depth, thickness and arc-width of the groove in the inlay,
such that the arcuately-shaped denture base is capable of attachment to the inlay by inserting the blade-shaped bottom edge of the arcuately-shaped denture base into the groove of the inlay and mating all surfaces of the blade-shaped bottom edge to the corresponding surfaces of the groove in the inlay,
whereby, upon fully mating, approximating into the negative replica counterpart of the arcuately-shaped denture base blade-shaped bottom edge to the inlay groove, a friction fit attachment occurs between the arcuately-shaped denture base and the inlay.

2. The method of using the two-part dental appliance of claim 1, comprising the steps:
obtaining a digital image of an edentulous region of a mandible of maxilla of a jaw of a patient using a CBCT (cone beam computed tomography) camera,
obtaining a digital image of the edentulous region of the mandible or maxilla of the jaw of the patient using an intraoral scanner,
digitally combining the digital image of the CBCT and the digital image of the intraoral scanner to obtain a 3D STL (stereolithography format) file of the edentulous region of the mandible or maxilla of the jaw of the patient,
using the 3D STL file to manufacture an arcuately-shaped denture base and an inlay corresponding to the edentulous region of the mandible or maxilla of the jaw of the patient,
using the 3D STL file to manufacture surgical guides corresponding to the edentulous region of the mandible or maxilla of the jaw of the patient, whereby the surgical guides limit the range of surgical tools that are used within the edentulous region of the mandible or maxilla of the jaw of the patient to prepare the mandible or maxilla of the jaw of the patient for installing the two-part dental appliance,
using the surgical guides to provide an incision within the edentulous region of the mandible or maxilla of the jaw of the patient, whereby an arcuate incision along an alveolar crest in the edentulous region of the mandible or maxilla of the jaw of the patient is made through the soft mucosal and gingival tissue,
using the surgical guides to provide an arcuate incision through the cortical bone along the alveolar crest of the edentulous region of the mandible or maxilla of the jaw of the patient,
using the surgical guides and surgical tools to create a channel within the cancellous tissue of the alveolar bone in the edentulous region of the mandible or maxilla of the jaw of the patient, thereby establishing osseo densification along the channel,
using the surgical guides and surgical tools to compact the cancellous tissue in the channel,
installing the inlay within the channel with the groove opening oriented to face in the coronal direction,
installing the arcuately-shaped denture base onto the inlay such that the blade-shaped bottom edge of the denture base mates within the groove of the inlay to make a friction fit attachment of the denture base to the inlay, and,
providing suturing of the gingival and mucosal tissue to surround the exposed denture base and initiate healing of the tissue.

3. The two-part dental appliance of claim 1 wherein the denture base includes a plurality of threaded holes extending from the top surface of the denture base to its blade-shaped bottom edge,
such that a plurality of screws can be inserted and threaded into the plurality of holes to extend and protrude the screws through the blade-shaped bottom of the denture base, causing the blade-shaped denture base to separate from the groove of the inlay.

4. The two part dental appliance of claim 1, wherein the inlay is provided with a glucose sensor and digital circuitry for measuring surrounding tissue glucose levels, and a communication port that communicates the glucose sensor with the surrounding tissue within the alveolar bone.
